(19) United States
(12) Patent Application Publication
(10) Pub. No.: US 2024/0374621 A1
(43) Pub. Date: Nov. 14, 2024

(54) CANNABINOID-HUMIC SUBSTANCES COMPOSITION AND MEHTHOD OF MAKING THE SAME

(71) Applicant: Hamed Ghannad, Kelowna (CA)

(72) Inventor: Faezeh DOUSTY, Kelowna, British Columbia (CA)

(21) Appl. No.: 18/690,394

(22) PCT Filed: Sep. 8, 2022
(86) PCT No.: PCT/CA2022/051343

§ 371 Date: Mar. 8, 2024

(30) Foreign Application Priority Data
Sep. 9, 2021 (CA) 3130284

(51) Int. Cl. A61K 31/00; A61K 9/00; A61K 31/202; A61K 31/353; B01D 11/02

(52) U.S. Cl. CPC A61K 31/658; A61K 9/0002; A61K 31/202; A61K 31/353; B01D 11/0203; B01D 11/0288

(57) ABSTRACT

A cannabinoid-humic substances composition is provided. The cannabinoid-humic substances composition includes a cannabinoid and humic substances perforated by voids. The cannabinoid is complexed with humic substances and fixed in the voids. A method for making the cannabinoid-humic substances composition is also provided. The method includes supplying (a) humic substances having a structure that is perforated by voids and (b) a cannabinoid. Once the cannabinoid is complexed with the humic substances, the cannabinoid-humic substances composition becomes water soluble.

### TECHNICAL FIELD

The present invention relates to a cannabinoid-humic substances composition and a method of making the same. In some embodiments, the present invention relates to a cannabinoid-humic substances composition that provides water-soluble cannabinoids. The cannabinoid-humic substances composition may be used in pharmaceutical formulations for human or veterinary use, natural health products, veterinary health products, topical cosmetics, beverages, or edibles. Some embodiments relate to a method of making a cannabinoid-humic substances composition, wherein humic substances are used to trap or fix cannabinoids in voids of the humic substances.

### BACKGROUND

There is limited prior art that discusses cannabinoids in the same context as humic substances.

Cannabinoids are chemical compounds that bind as direct agonists to cannabinoid receptors in the body and brain. The first discovered cannabinoids are those derived from Cannabis sativa. To date, more than 100 different cannabinoids have been identified in the cannabis plant. Some notable ones are tetrahydrocannabinol (THC), cannabidiol (CBD), cannabinol (CBN), cannabigerol (CBG), and cannabichromene (CBC). Cannabinoids may be extracted from the cannabis plant or synthetically made.

The two main cannabinoids are THC and CBD. THC has received much attention for being the main psychoactive component of the cannabis plant. The psychoactive effects of THC are due to its ability to activate type-1 cannabinoid (CB₁) receptors that are located mainly in the central nervous system. CBD does not appear to have any intoxicating effects such as those caused by THC, but may have effects on anxiety and act as an anti-psychotic. Further, cannabinoids have been used to relieve chronic pain, inflammation, depression, epilepsy, chemotherapy-induced nausea and vomiting, and anorexia and weight loss associated with HIV/AIDS.

Cannabinoids may be obtained from trichrome by supercritical carbon dioxide (CO₂) extraction. Supercritical CO₂ has been widely used as an extraction solvent because it is abundant, inexpensive, non-toxic, non-flammable, relatively chemically inert, and forms at almost room temperature (at 31° C.). CO₂ becomes supercritical when it is compressed to a pressure and elevated to a temperature greater than that of the critical point. Supercritical CO₂ exhibits properties of both a liquid and a gas. For example, supercritical CO₂ has a liquid-like density and gas-like diffusivity and viscosity.

Humic substances are formed in nature and arise from decayed or partially decayed organic material. Humic substances include humic acid(s), fulvic acid(s), and humin. The terms “fulvic acid(s)”, “humic acid(s)”, and “humin” do not refer to discrete chemical compounds. Each of these terms includes a wide variety of compounds of varying molecular weight, solubility, and spectral characteristics. In general terms, the distinction of humic substances as between the categories of humic acid(s), fulvic acid(s), and humin is based on their solubilities in acidic and alkaline aqueous solutions. In particular, humin is insoluble under both acidic and alkaline conditions, and have larger molecular weights, generally greater than about 100,000 Daltons, than both humic acid(s) and fulvic acid(s). Humic acid(s) is soluble under alkaline conditions, but is insoluble in acidic conditions. The molecular weights of humic acid(s) ranges from a lower limit of about 2,500-12,500 Daltons to an upper limit of about 75,000-300,000 Daltons. Fulvic acid(s) is soluble under both acidic and alkaline conditions. Fulvic acid(s) is the lightest fraction of humic substances. The molecular weights of fulvic acid(s) range from a lower limit of about 250-1,000 Daltons to an upper limit of about 2,500-12,500 Daltons.

Humic substances have been used in agricultural applications as fertilizers and soil additives. For example, humic acid supplementation has been shown to affect the cannabinoid content in cannabis plants. See for example Impact of N, P, K, and Humic Acid Supplementation on the Chemical Profile of Medical Cannabis (Cannabis sativa L) by Bernstein et al., in Frontiers in Plant Science, 2019, Volume 10.

Other than the agricultural applications of humic substances, it is not known whether humic substances might be useful to overcome the many shortcomings of the cannabinoid-related prior art. For example, cannabinoids are highly lipophilic. Cannabinoids are soluble in fats and alcohols but are not soluble in water. The high lipophilicity has placed some constraints on the use applications of cannabinoids in pharmaceutical formulations, natural health products, topical cosmetics, and/or beverages. The high lipophilicity is also associated with in efficient and unpredictable bioavailability. To this end, there is a desire for a composition that provides water-dissolvable cannabinoids.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

This invention has several aspects. These include without limitation:
a cannabinoid-humic substances composition;
a method of making a cannabinoid-humic substances composition; and
an apparatus for making a cannabinoid-humic substances composition.

In one aspect, a new and useful cannabinoid-humic substances composition is provided. Without being bound by theory, some advantages that may be provided by some embodiments of the cannabinoid-humic substances composition include:
the cannabinoid-humic substances composition may provide water-soluble cannabinoids—the composition may display hydrophilic properties permitting a wide array of use cases;
the cannabinoid-humic substances composition may delay cannabinoid degradation during storage of the cannabinoid-humic substances composition;
the cannabinoid-humic substances composition may improve cannabinoid bioavailability when the cannabinoid-humic substances composition is consumed via routes other than intravenous; and
the cannabinoid-humic substances composition may be used in pharmaceutical formulations for human or veterinary use, dietary supplement, natural health products, veterinary health products, topical cosmetics, food and beverage, or edibles.

The cannabinoid-humic substances composition contains cannabinoid(s) and humic substances. The humic substances are perforated by voids and the cannabinoid(s) is trapped (or fixed) in the voids.

The voids may be of any suitable dimensions for trapping the cannabinoid(s).

The cannabinoid(s) may complex with the humic substances.

The voids each may provide a polarity that is complementary to that of the cannabinoid(s) trapped in the voids. The cannabinoid(s) is immobilized in the voids by the complementary polarity between the cannabinoid(s) and the voids.

The cannabinoid(s) may be THC, CBD, CBN, CBG, CBC, or a combination of different cannabinoids.

The humic substances may be humic acids, fulvic acids, and/or humin.

The cannabinoid-humic substances composition may provide a cannabinoid release rate of 75% to 95% within the first 60 minutes to 150 minutes after the cannabinoid-humic substances composition is ingested.

The amount of cannabinoid in the cannabinoid-humic substances composition may be in the range of 1% to 30% on a dry matter basis.

The cannabinoid-humic substances composition may include flavoring agents, glidants, and/or super disintegrants.

Another aspect of the invention is directed to use of humic substances for complexing with a cannabinoid. The complexation reaction leads to the cannabinoid being trapped (or fixed) in voids of the humic substances.

Another aspect of the invention is directed to a method of making a cannabinoid-humic substances composition.

The method includes supplying (i) humic substances perforated by voids and (ii) a cannabinoid. The cannabinoid is then trapped in the voids to thereby provide the cannabinoid-humic substances composition.

In some embodiments, the cannabinoid is dissolved in supercritical CO₂ to form a cannabinoid-supercritical CO₂ solution. The humic substances are infused with the cannabinoid-supercritical CO₂ solution. Upon a pressure drop, the cannabinoid separates from supercritical CO₂ and is deposited in the voids. Supercritical CO₂ is transformed into gaseous CO₂ and is removed from the cannabinoid-humic substances composition. The pressure drop may be a sudden pressure reduction or a gradual pressure reduction over a period of time.

When the humic substances are infused with the cannabinoid-supercritical CO₂ solution, the cannabinoid may complex with the humic substances.

The infusion step may occur by soaking the humic substances in the cannabinoid-supercritical CO₂ solution or by flowing the cannabinoid-supercritical CO₂ solution through the humic substances.

The intermolecular forces between the cannabinoid and the humic substances may provide attraction between the cannabinoid and the voids.

To increase the available voids in the humic substances, the humic substances may be washed with an organic solvent, such as food-grade alcohol, ethanol, heptane, and/or hexane.

Another aspect of the invention provides a system for making a cannabinoid-humic substances composition. The system has an infusion chamber in selective fluid communication with a cannabinoid supply source and a humic substances supply source. The infusion chamber (vessel, device, or apparatus) has a first inlet in selective fluid communication with the cannabinoid supply source. The first inlet has an inlet passage through which supercritical CO₂ carrying a cannabinoid is to enter the infusion chamber. Upon a pressure drop in the infusion chamber, the cannabinoid separates from supercritical CO₂ and supercritical CO₂ is transformed to gaseous CO₂. The infusion chamber has a second inlet in selective fluid communication with the humic substances supply source, the second inlet through which humic substances are to enter the infusion chamber. The infusion chamber has a first outlet having an outlet passage through which the gaseous CO₂ is to exist the infusion chamber and to enter the supercritical phase for recirculation through the infusion chamber. The infusion chamber has a second outlet for collecting cannabinoid-humic substances composition.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a flow diagram of an example embodiment of a method of making a cannabinoid-humic substances composition.

FIG. 2 is a schematic diagram of an example system for carrying out the method of FIG. 1.

FIG. 3 is a photograph of a cannabinoid-humic substances composition made according to one example embodiment.

FIG. 4 is a photograph of a weighing dish containing a measured amount of a CBD isolate and a measured amount of humic substances.

FIG. 5 is a photograph of a solution of water and the cannabinoid-humic substances composition of FIG. 3.

FIG. 6 is a photograph of a solution of water and a physical mixture of the CBD isolate and humic substances of FIG. 4.

FIG. 7A is a graph showing the FTIR absorbance spectra for the cannabinoid-humic substances composition of FIG. 3.

FIG. 7B is a graph showing the FTIR absorbance spectra for the CBD isolate of FIG. 4.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Various chemical substances are described herein. Such chemical substances may not be absolute pure. Instead, such substances may have a purity of at least about 50%, at least about 60%, at least about 70%, at least about 80%, at least about 90%, at least about 95%, at least about 97%, at least about 98%, or at least about 99% pure. The expression “substantially pure” means a purity of greater than 95%. For example, “substantially pure” cannabinoid preparation means that the preparation having a chromatographic purity of greater than 95%, 96%, 97%, 98% or 99%.

As used herein, the terms “about” or “approximately” mean a value within +/−10% of the stated value unless specified otherwise. Either one of these terms “about” or “approximately” connotes that strict compliance with the numeric value recited is not critical. Some variation is permissible and still within the scope of the various embodiments described herein.

As used herein, “cannabis” includes all plants belonging to the genus Cannabis, which includes hemp and marijuana, and includes all Cannabis species such as Cannabis indica, Cannabis sativa, Cannabis ruderalis, and other such Cannabis species as may be identified or reclassified from time to time, and further includes industrial hemp, and the like.

As used herein, “cannabinoids” are chemical compounds that bind as direct agonists to cannabinoid receptors in the body and brain. Cannabinoids may be extracted from the cannabis plant or synthetically made. Some notable ones are tetrahydrocannabinol (THC), cannabidiol (CBD), cannabinol (CBN), cannabigerol (CBG), and cannabichromene (CBC).

As used herein, the term “flavoring agents” refers to any substance that may be added to create an additional taste or flavour. Flavouring agents may be added to help in masking unpleasant tastes. Flavoring agents may include aromatic oils (e.g., caraway, clove, lemon, spearmint, rose, and peppermint); sweetening agents (e.g. sucrose and sorbitol) ginger; raspberry; maltol; syrups (e.g., citric acid, sarsaparilla, and cherry); glycerin; cocoa; licorice; vanillin; and ethyl vanillin.

As used herein, the term “glidants” refers to any substance that may be added to enhance the flowability of a formulation. Glidants may include colloidal silica, magnesium silicate, and magnesium stearate.

As used herein, the term “super disintegrants” refers to any substance that may be added in a formulation to facilitate dissolution or release the active ingredients contained in the formulation. Super disintegrants may include cross-linked polyvinyl pyrrolidone, croscarmellose sodium, and sodium starch glycolate.

As used herein, “insoluble” means that not more than 0.1 g will dissolve in 100 ml of water. The term “sparingly soluble” means that than not more than 3.3 g will dissolve in 100 ml of water. The term “soluble” means that about or more than 3.3 g will dissolve in 100 ml of water.

The inventor has found that humic substances, including humic acids and fulvic acids, have a flexible structure perforated by voids that can trap cannabinoids. Based on this, the inventor has invented new and useful cannabinoid-humic substances composition and a method of making the same.

In some embodiments, the cannabinoid-humic substances composition:
provides water-dissolvable/soluble cannabinoids;
delays cannabinoid degradation—in comparison with a composition had the cannabinoids not been trapped in the voids of the humic substances;
improves cannabinoid bioavailability when the cannabinoid-humic substances composition is consumed via routes other than intravenous; and/or
may be used in pharmaceutical formulations for human or veterinary use, natural health products, veterinary health products, topical cosmetics, beverages, or edibles.

A cannabinoid-humic substances composition according to a first embodiment of the present invention includes (i) a cannabinoid and (ii) humic substances. The humic substances have a structure perforated by voids. The cannabinoid is trapped (or fixed) in the voids. The cannabinoid may have a polarity that is complementary to that of the voids. The cannabinoid may complex with the humic substances.

The amount of humic substances in the cannabinoid-humic substances composition is in the range of about 70% to about 99% (w/w on a dry matter basis), including any value therebetween, e.g. about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, and 98%. The ratio of humic substances to cannabinoid(s) may vary depending on the desired use applications (e.g. pharmaceutical formulations for human or veterinary use, natural health products, veterinary health products, topical cosmetics, beverages, and edibles), the chemical and physical characteristics of the humic substances, the chemical and physical characteristics of the cannabinoid(s), and the conditions used to prepare the cannabinoid-humic substances composition.

For example, the cannabinoid-humic substances composition may be used in a pharmaceutical formulation where cannabinoid(s) is an active ingredient. Such pharmaceutical formulation may require a certain specific ratio of cannabinoid(s) to humic substances.

The available voids in the humic substances may control the cannabinoid loading capacity. In other words, the available voids may influence the amount of cannabinoid(s) that may be fixed in the humic substances. For example, with more voids available, the humic substances could theoretically trap more cannabinoid(s) in such voids. To increase the available voids in the humic substances, the humic substances may be washed by an organic solvent, including food-grade alcohol, ethanol, heptane, and/or hexane.

The characteristics of the cannabinoid(s) may influence the ratio of the cannabinoid(s) trapped in the cannabinoid-humic substances composition to the humic substances. For example, the cannabinoid(s) may have a polarity that is complementary to that of the voids. Complementary polarity would enable intermolecular forces between the cannabinoid(s) and the voids to attract each other. The cannabinoid(s) with a polarity would attract voids with an opposite polarity. This may lead to more stable entrapment of the cannabinoid(s) in the voids. This may also lead to a higher ratio of the cannabinoid(s) trapped in the cannabinoid-humic substances composition to the humic substances.

The conditions, including the operating pressure and temperature, supercritical CO₂ flow scale, cannabinoid(s)-to-humic substances ratio, recovery conditions (precipitation), and other mass transfer parameters, used to prepare the cannabinoid-humic substances composition may be changes and/or optimized so that a certain specific ratio of the cannabinoid(s) trapped in the cannabinoid-humic substances composition to the humic substances can be achieved.

A sufficient proportion of humic substances should be present in order to trap cannabinoids. An excessive proportion of humic substances in the cannabinoid-humic substances composition may not be desirable if that would negative affect the use applications of the cannabinoid-humic substances composition.

Humic substances may be humic acids, fulvic acids, and/or humin.

Humic substances may be subject to a pre-processing step. For example, to increase the available voids in the humic substances, the humic substances may be washed by an organic solvent, including food-grade alcohol, ethanol, heptane, and/or hexane.

The amount of the cannabinoid(s) present in the cannabinoid-humic substances composition is between about 1% to about 30% (w/w on a dry matter basis), including any value therebetween, e.g. about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, and 29%.

The cannabinoid(s) can be any cannabinoid(s) isolated from the cannabis plant or made synthetically.

The cannabinoid-humic substances composition may include a flavoring agent, a glidants, and/or a super disintegrants.

The cannabinoid-humic substances composition may be water soluble. Without being bound by theory, this water-soluble property may result from a change in the aromatic hydrophobic structure of the cannabinoid(s). Once the cannabinoid(s) is complexed with humic substances, the structure of the cannabinoid(s) may change to an expanded hydrophilic structure.

The cannabinoid-humic substances composition may improve cannabinoid's bioavailability. Without being bound by theory, the improved bioavailability may be because humic substances act as carriers and deliver cannabinoid(s) to the bloodstream.

The cannabinoid-humic substances composition has a higher dissolution/release rate. The cannabinoid-humic substances composition may provide a cannabinoid release rate of 75% to 95% within the first 60 minutes to 150 minutes after the cannabinoid-humic substances composition is ingested.

The cannabinoid-humic substances composition may be used directly, or mixed or dissolved in other carriers. For example, the cannabinoid-humic substances composition may be formulated together with one or more pharmaceutically acceptable carriers (additives) and/or diluents (collectively referred to herein as pharmaceutical carriers) suitably selected with respect to the intended form of administration, e.g., oral tablets, capsules, and syrups, and consistent with conventional pharmaceutical practices. Optionally, one or more additional therapeutic agents may be added. In this regard, the cannabinoid-humic substances composition may be used for medicinal purposes.

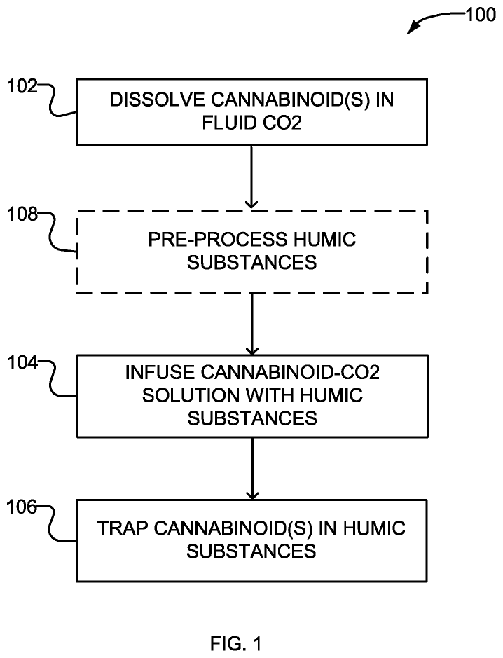

FIG. 1 shows an example embodiment of a method 100 of making a cannabinoid-humic substances composition.

Briefly, method 100 deploys the solubility of cannabinoid(s) in super- and/or sub-critical CO₂, e.g. typically above the temperature of 30° C. and above the pressure of 1,000 psi (e.g. in the pressure range of 1400 psi and 3100 psi). Cannabinoid(s) is dissolved in and carried by super- and/or sub-critical CO₂. Upon a pressure drop, the cannabinoid(s) separates from the super- and/or sub-critical CO₂ and precipitates in the voids of the humic substances. The cannabinoid(s) complexes with the humic substances and is trapped in the voids of the humic substance. Also upon the pressure drop, the super- and/or sub-critical CO₂ may enter a gaseous phase and is removed without leaving any residue in the cannabinoid-humic substances composition. The removed CO₂ may be recycled and reused. The cannabinoid-humic substances composition may be formed at the bottom of an infusion apparatus (e.g. a chamber, a vessel, or any other suitable devices). The cannabinoid-humic substances composition may be collected and further processed, e.g. to be homogenized.

At step 102, a cannabinoid is dissolved in super- or sub-critical CO₂ to form a cannabinoid-CO₂ solution. In some embodiments, the conditions at step 102, including the operating temperature and pressure of the super- or sub-critical CO₂, the flow rate, and solvent-to-feed ratio, are selected to promote the solubility of cannabinoid in the super- or sub-critical CO₂.

Step 102 may be part of an extraction process using supercritical or subcritical carbon dioxide (CO₂) as a solvent to extract cannabinoid(s). The CO₂ used in step 102 may have a purity of 99% and higher. Generally speaking, CO₂ is an effective solvent for extracting cannabinoids.

In some embodiments, supercritical CO₂-assisted atomization/spray drying may be used. For example, water may be used as a co-solvent and/or a wetting agent. Supercritical CO₂-assisted atomization/spray drying may be used when water is used to increase complexation efficiency. In some other embodiments, thermos-liable cannabinoid(s) may be of interest. Supercritical CO₂-assisted atomization/spray drying may be used to reduce the size of thermos-liable cannabinoid(s) droplets and facilitate the drying to occur at a lower temperature.

Other modifications of supercritical CO₂ may include:
Rapid Expansion of Supercritical Solution (RESS);
Rapid Expansion of a Supercritical Solution into a Liquid Solvent (RESOLV);
Gaseous Antisolvent (GAS);
Particles by Compressed Antisolvent (PCA); and
Supercritical Antisolvent (SAS).

A co-solvent may be used to assist the solubility of cannabinoid(s) in super- or sub-critical CO₂. The co-solvent may be acid(s) or low molecular-weight alcohols.

At step 104, the cannabinoid-CO₂ solution is infused with humic substances. For example, the cannabinoid-CO₂ solution may flow through and saturate humic substances. Depending on the chemical properties of the interested cannabinoid(s), the infusion process may occur under static (e.g. a batch operation) or dynamic conditions (e.g. a continuous operation).

The conditions at step 104 are selected to promote infusion and complexation of the interested cannabinoid(s) with humic substances. Certain physical and/or chemical properties of CO₂, e.g. its compressibility, diffusivity, and evaporation rate, permits optimization and modification of the conditions at step 104 to be achieved.

Infusion time may vary depending on the chemical and physical properties of the interested cannabinoid(s). In some embodiments, the infusion time may be adjusted to match the time duration that it would take to reach equilibrium.

Without being bound by theory, humic substances may form complexes with cannabinoid(s) and the cannabinoid(s) is further immobilized within the voids by complementary polarity between the cannabinoid and the voids.

At step 106, the cannabinoid becomes trapped in the voids of the humic substances where there is a pressure drop. For example, the pressure drop may cause (i) the cannabinoid to separate from supercritical CO₂ and to precipitate/become trapped in the voids of the humic substances and (ii) the supercritical CO₂ to evaporate or enter a regular liquid phase (i.e. neither a supercritical nor a subcritical phase). The removed CO₂ may be recycled and reused as solvent to dissolve or extract cannabinoid(s). In some embodiments, the pressure is reduced from a pressure range of about 1900 psi and 3800 psi to pressure range of about 500 psi and 1000 psi. In some embodiments, the pressure is reduced to atmospheric pressure. In some embodiments, the pressure is reduced to below 550 psi. In some embodiments, the pressure drop may be sudden. In some other embodiments, the pressure drop may be gradual over a period of time.

Before step 104, if necessary or desirable, the humic substances may be subject to a pre-processing step 108. For example, the humic substances may be washed by an organic solvent to increase the available voids in the humic substances.

In some embodiments, steps 102 and 104 may be carried out simultaneously. For example, humic substances may be loaded into a vessel and cannabinoid(s) may be loaded into a tube. The vessel and tube are in fluid communication with each other. CO₂ is pumped into the vessel and the tube. Temperature and pressure in the vessel and the tube change so that regular CO₂ becomes supercritical CO₂. The supercritical CO₂ dissolves the cannabinoid(s) and carries the dissolved cannabinoid(s) to the humic substances so that dissolved cannabinoid(s) is diffused into the voids present in the humic substances and complexes with the humic substances.

The inventor has observed that as the density of the supercritical CO₂ increases, a higher level of cannabinoid(s) enter into the voids of the humic substances. However, when the density of the supercritical CO₂ reaches a threshold, the inventor has observed that the hydrophobic interactions between the cannabinoid(s) and the supercritical CO₂ become increase, which could limit the amount of cannabinoid(s) enter and/or remain in the voids of the humic substances. In some embodiments, the pressure in the vessel may be modified to control the density of the supercritical CO₂ so that the interactions between supercritical CO₂ and cannabinoid(s) would not interfere with the complexation between cannabinoid(s) and humic substances.

The inventor has also observed that the density of the supercritical CO₂ is dependent on temperature. The density the supercritical CO₂ decreases when its temperature increases. The inventor has observed that when the temperature of supercritical CO₂ reaches a threshold, cannabinoid-humic substances complexation efficiency decreases. Without being bound by theory, this could be because a high temperature would increase molecular movement and reduce the rate of entrance of cannabinoid(s) into the voids of humic substances.

In some embodiments, a co-solvent may be used, for example to improve the complexation efficiency between cannabinoid(s) and humic substances. For example, water may be a co-solvent. The addition of water to the supercritical CO₂ would decrease the hydrophobic interactions between the cannabinoids(s) and the supercritical CO₂. This decrease in the hydrophobic interactions may lead to easier entrance of cannabinoid(s) into the voids of humic substances. Additionally, when water is used to wet the humic substances, the wetted surface of humic substances may promote surface contact with cannabinoid(s), thereby improving the complexation efficiency.

In some embodiments, a wetting agent may be used, for example to improve the complexation efficiency between cannabinoid(s) and humic substances.

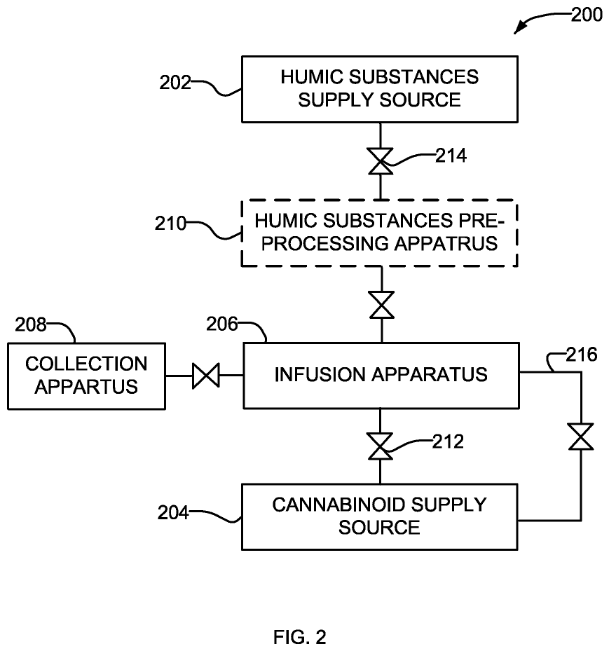

Method 100 may be carried out in a system 200 for making a cannabinoid-humic substances composition. With reference to FIG. 2, system 200 has a humic substances supply source 202, a cannabinoid supply source 204, an infusion apparatus 206.

The lines connecting the components shown in FIG. 2 may represent hoses, pipes, tubes, fluid transfer lines, or any other like device(s) for fluid conveyance. Such fluid conveyance devices may be manufactured from stainless steel and/or aeronautic/medical grade aluminium, and may also be lined with polytetrafluoroethylene. The size, shape, length, inner diameter, outer diameter, and the like of such fluid conveyance devices may be chosen according to various design choices.

Humic substances supply source 202 supplies humic substances, such as humic acids, fulvic acids, and/or humin, to system 200. In some embodiments, humic substances supply source 202 may be an apparatus for extractive humic substances from humus material, as described in U.S. Pat. No. 7,896,944.

Cannabinoid supply source 204 supplies cannabinoid(s) to system 200. In some embodiments, cannabinoid supply source 202 is a supercritical fluid extraction apparatus that permits the extraction/dissolving of cannabinoid(s) by super- or sub-critical CO₂. The supercritical fluid extraction apparatus may be powered by an air compressor, a CO₂ supply that may include one or more tanks of CO₂, a heating element, an extraction chamber, an air cooling device, and a collection chamber.

Infusion apparatus 206 is in selective fluid communication with humic substances supply source 202 and cannabinoid supply source 204. Suitable valves 212, 214 may be present to maintain the respective pressures within humic substances supply source 202, cannabinoid supply source 204, and infusion apparatus 206. Suitable valves 212, 214 may also control the flow of fluids (i) from humic substances supply source 202 to infusion apparatus 206 and (ii) from cannabinoid supply source 204 to infusion apparatus 206.

For example, infusion apparatus 206 has a first inlet (not shown) and a second inlet (not shown). The first inlet of infusion apparatus 206 is connected to humic substances supply source 202 and humic substances are moved from humic substances supply source 202 to infusion apparatus 206 via first inlet. The second inlet of infusion apparatus 206 is connected to cannabinoid supply source 204 and a pump (not shown) may be used to move the super- or sub-critical CO₂ containing dissolved cannabinoid(s) from cannabinoid supply source 204 to infusion apparatus 206 via second inlet.

In infusion apparatus 206, the super- or sub-critical CO₂ containing dissolved cannabinoid(s) is infused with the humic substances. The infusion process may permit complexation between cannabinoid(s) and humic substances. Once infusion has occurred, the pressure in infusion apparatus 206 drops by an amount that may result in the CO₂ entering a gas phase or a regular liquid phase (i.e. neither a supercritical nor a subcritical phase). CO₂ may be recycled back to cannabinoid supply source 204 via line 216 so that the recycled CO₂ can be used to extract/dissolve additional cannabinoid(s). Also upon the pressure drop, the cannabinoids precipitate and become trapped in voids of the humic substances, to thereby form a cannabinoid-humic substances composition.

The cannabinoid-humic substances composition may exit from infusion apparatus 206 and enter into a collection apparatus 208.

Humic substances may be pre-processed in humic substances pro-processing apparatus 210 wherein the humic substances may be washed by an organic solvent to increase the available voids in the humic substances.

### EXAMPLES

Specific examples are described below, which are illustrative and not limiting in nature.

### Example 1.0—Making of Cannabinoid-Fulvic Acids Composition

Fulvic acids were loaded into a stainless-steel vessel.

CBD isolate was loaded into a tube before supercritical CO₂ was flowed into the tube.

Supercritical CO₂ was flowed into the tube to dissolve the CBD isolate. The supercritical CO₂ was compressed to a pressure of about 30 MPa and at a temperature of about 350° K.

The supercritical CO₂ carrying dissolved CBD was flowed into the stainless-steel vessel containing fulvic acids. The supercritical CO₂ carrying dissolved CBD saturated the fulvic acids. The pressure and temperature in the stainless-steel vessel were about 30 MP and about 350° K, respectively.

After a reaction time of 4 hours, the pressure in the stainless-steel vessel was decreased to atmospheric pressure.

### Example 1.5—Making of Cannabinoid-Humic Substances Composition

A mixture of fulvic acids and humic acids (50% w/w on a dry matter basis) was loaded into a stainless-steel vessel.

CBD isolate was loaded into a tube before supercritical CO₂ was flowed into the tube.

Supercritical CO₂ was flowed into the tube to dissolve the CBD isolate. The supercritical CO₂ was compressed to a pressure of about 30 MPa and at a temperature of about 350° K.

The supercritical CO₂ carrying dissolved CBD was flowed into the stainless-steel vessel containing humic substances. The supercritical CO₂ carrying dissolved CBD saturated the humic substances. The pressure and temperature in the stainless-steel vessel were about 30 MP and about 350° K, respectively.

After a reaction time of 4 hours, the pressure in the stainless-steel vessel was decreased to atmospheric pressure.

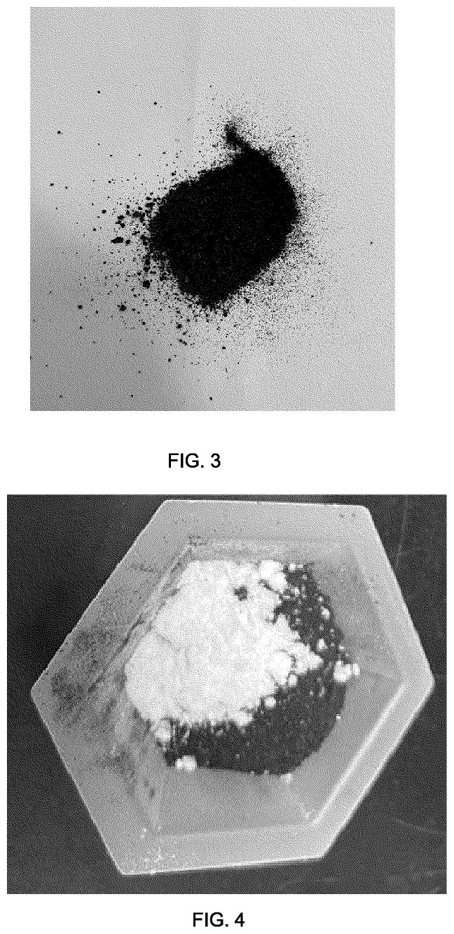

A cannabinoid-humic substances composition, shown in FIG. 3, was collected from the button of the stainless-steel vessel.

### Example 2.0—Water Solubility Study

The cannabinoid-humic substances composition obtained from Example 1.5 was shown to have a higher dissolution/release rate.

2.5 g of all samples was placed in 50 ml of water at 37° C. and stirred with magnetic stick. During the dissolution process, sub samples were collected at different intervals, filtered by a 0.45 membrane.

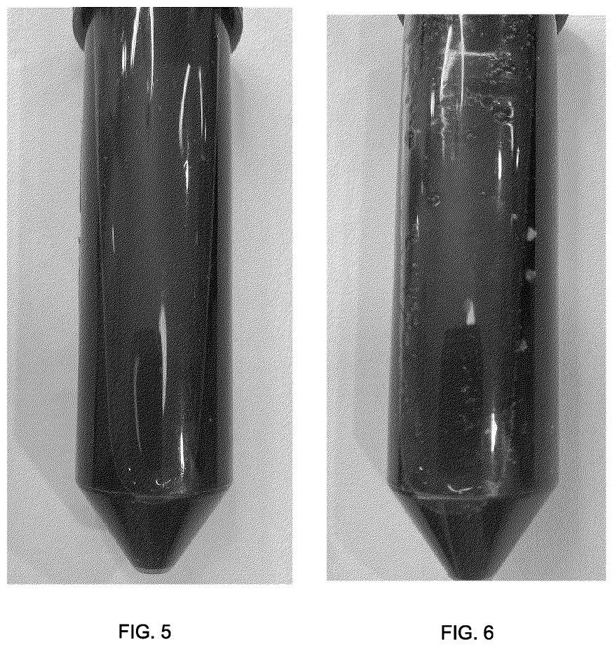

The inventor observed that the 2.5 g of the composition obtained from Example 1.5 was completely dissolved in 50 ml of water at room temperature. See FIG. 5.

The inventor observed that the 2.5 g of unprocessed physical mixture was not completely dissolved in 50 ml of water at room temperature. See FIG. 6.

The composition obtained from Example 1.5 showed a higher dissolution rate than the CBD isolate and the simple mix of CBD isolate and humic substances.

The filtrates were injected to an Ultra-high-performance liquid chromatography (UPLC) and the cannabinoid contents were analyzed by a UV-VIS detector (LaChrom Elite L-2420).

### Example 3.0—Complexation Efficiency Study

The cannabinoid-humic substances composition obtained from Example 1.5 is shown to be more stable. The inventor believes that the complexation of cannabinoids in the voids of humic substances prevents degradation of cannabinoids.

A complexation efficiency study was carried out using attenuated total reflectance Fourier transform infrared spectroscopy (ATR-FTIR).

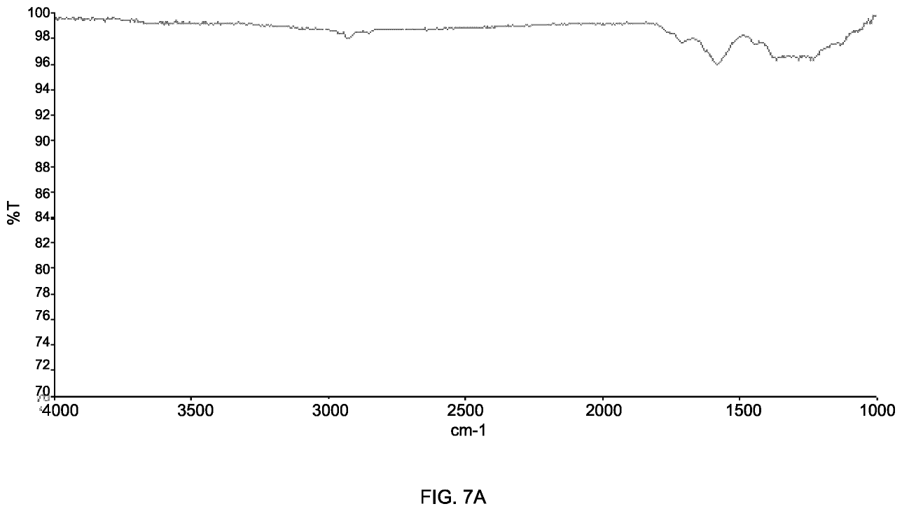

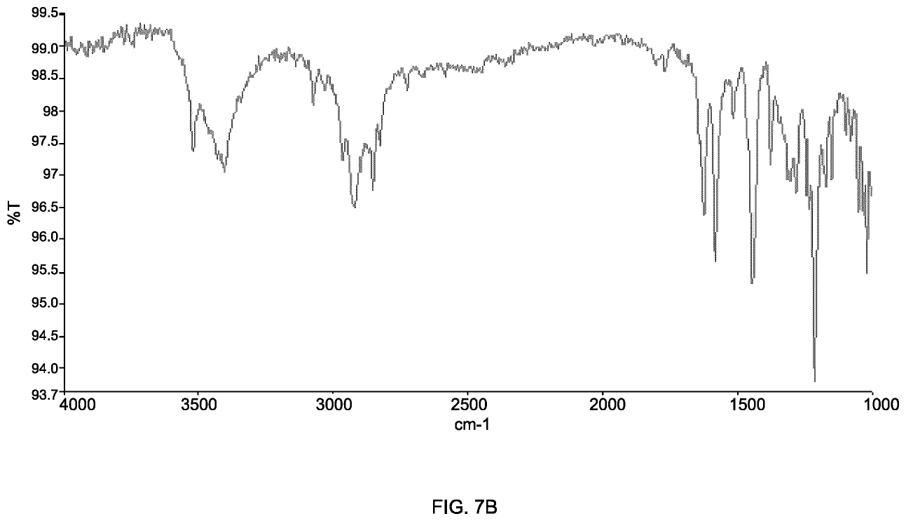

FIG. 7A is a graph showing the FTIR absorbance spectra for the cannabinoid-humic substances composition obtained from Example 1.5. FIG. 7B is a graph showing the FTIR absorbance spectra for the CBD isolate.

A comparison of FIGS. 7A and 7B show that the index peaks of the CBD isolate are missing from the cannabinoid-humic substances composition. The absence of such index peaks suggest that the cannabinoid is complexed with humic substances.

### Example 4.0—Ultraviolet-Visible (UV-VIS) Spectroscopy

To confirm the formation of cannabinoid-humic substances complex, UV-VIS spectroscopy can be used.

The UV-VIS spectra of humic substances, pure cannabinoids, the control, and the inclusion complexes were collected on a UV-1800 Shimadzu UV-Vis Spectrophotometer with a resolution of 0.5 nm.

The inventor suspects that significant changes to the UV absorption spectrum of the cannabinoids would be observed.

### Example 5.0—Scanning Electron Microscopy (SEM)

The SEM images of humic substances, pure cannabinoids, the control, and the inclusion complexes may be collected.

The inventor suspects that SEM image of the inclusion complex of cannabinoids would show irregular shapes compared to the ones from pure cannabinoids, which suggests different morphologies as the results of complexation of cannabinoids within the body of humic substances.

### Example 6.0—Ultra-High-Performance Liquid Chromatography (UPLC) System with a Diode Array Detector (DAD)

An Agilent UPLC/DAD system was used for the assay of cannabinoids content.

Stock solutions of cannabinoids were prepared in methanol. Serial dilutions were prepared from the stock solution to equal six concentrations between 0.4 mg/ml and 1.2 mg/ml and the linear curve were prepared from triplicate injections of solutions. A Poroshell 120 CE-C18 was used as the column and either a 7-minute isocratic run or a 30-minute gradient run were used to measure the cannabinoid contents depending on the cannabinoids of interest. The mobile phase was acetonitrile and phosphate buffer at pH of 6.

The results confirm the inclusion yields of more than 90% for the cannabinoids contents.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1-33. (canceled)

34. A cannabinoid-humic substances composition comprising:
a cannabinoid; and
humic substances perforated by voids;
wherein the cannabinoid is fixed in the voids.

35. The composition of claim 34, wherein the cannabinoid complexes with the humic substances.

36. The composition of claim 34, wherein the voids each provide a polarity that is complementary to that of the cannabinoid.

37. The composition of claim 36, wherein the cannabinoid is immobilized in the voids by the complementary polarity between the cannabinoid and the voids.

38. The composition of claim 34, wherein the composition is water soluble.

39. The composition of claim 34, wherein the cannabinoid is tetrahydrocannabinol (THC), cannabidiol (CBD), cannabinol (CBN), cannabigerol (CBG), cannabichromene (CBC), or a combination of thereof.

40. The composition of claim 34, wherein the humic substances comprise humic acids, fulvic acids, or humin.

41. The composition of claim 34, wherein the cannabinoid-humic substances composition provides a cannabinoid release rate of 75% to 95% within the first 60 minutes to 150 minutes after the cannabinoid-humic substances composition is ingested.

42. The composition of claim 34, further comprising flavoring agents, glidants, or super disintegrants.

43. Use of humic substances for complexing with a cannabinoid, wherein:
the cannabinoid is fixed in voids of the humic substances;
the voids each provide a polarity that is complementary to that of the cannabinoid; and
the cannabinoid is immobilized in the voids by the complementary polarity between the cannabinoid and the voids.

44. A method of making a cannabinoid-humic substances composition, the method comprising:
supplying humic substances having a structure that is perforated by voids;
supplying a cannabinoid;
dissolving the cannabinoid in supercritical CO₂ to form a cannabinoid-supercritical CO₂ solution;
infusing the humic substances with the cannabinoid-supercritical CO₂ solution; and
upon a pressure drop:
precipitating the cannabinoid in the voids to fix the cannabinoid in the voids to provide the cannabinoid-humic substances composition; and
removing CO₂ as gaseous CO₂.

45. The method of claim 44, further comprising:
soaking the humic substances in the cannabinoid-supercritical CO₂ solution.

46. The method of claim 44, further comprising:
flowing the cannabinoid-supercritical CO₂ solution through the humic substances.

47. The method of claim 44, when the humic substances are infused with the cannabinoid-supercritical CO₂ solution, the cannabinoid complexes with the humic substances.

48. The method of claim 44, wherein intermolecular forces between the cannabinoid and the humic substances provide attraction between the cannabinoid and the voids.

49. The method of claim 44, further comprising:
washing the humic substances with an organic solvent.

50. The method of claim 49, wherein the organic solvent comprises a food-grade solvent and the food-grade solvent comprises alcohol, ethanol, heptane, or hexane.

51. The method of claim 44, wherein the cannabinoid is tetrahydrocannabinol (THC), cannabidiol (CBD), cannabinol (CBN), cannabigerol (CBG), cannabichromene (CBC), or a combination thereof.

52. The method of claim 44, wherein the humic substances comprise humic acids, fulvic acids, or humin.

53. A system for making a cannabinoid-humic substances composition, the system comprising:
an infusion chamber in selective fluid communication with a cannabinoid supply source and a humic substances supply source;
the infusion chamber comprising:
a first inlet in selective fluid communication with the cannabinoid supply source, wherein:
the first inlet comprises an inlet passage through which supercritical CO₂ carrying a cannabinoid is to enter the infusion chamber; and
upon a pressure drop in the infusion chamber, the cannabinoid separates from supercritical CO₂ and supercritical CO₂ is transformed to gaseous CO₂;
a second inlet in selective fluid communication with the humic substances supply source, the second inlet through which humic substances are to enter the infusion chamber;
a first outlet comprising an outlet passage through which the gaseous CO₂ is to exist the infusion chamber and to enter the supercritical phase for recirculation through the infusion chamber; and
a second outlet for collecting cannabinoid-humic substances composition.
